(19) United States
(12) Patent Application Publication
(10) Pub. No.: US 2023/0149748 A1
(43) Pub. Date: May 18, 2023

(54) Head and/or Neck-Mounted Aerosol-Based Respiratory Protection Device

(71) Applicant: Raimund Naschberger, Innsbruck (AT)

(72) Inventor: Raimund Naschberger, Innsbruck (AT)

(21) Appl. No.: 17/919,465

(22) PCT Filed: Apr. 17, 2021
(86) PCT No.: PCT/EP2021/060005

§ 371 Date: Oct. 17, 2022

(30) Foreign Application Priority Data
Apr. 17, 2020 (WO) 20170236.2
Apr. 19, 2020 (WO) 20170268.5

(51) Int. Cl. A62B 18/00

(52) U.S. Cl. CPC A62B 18/003

(57) ABSTRACT

The invention refers to a head and/or neck-mounted respiratory protection device (10) comprising a body-wearable elongated mount (12) for attaching the respiratory protection device (10) to the head and/or neck of a user, a reservoir (14) for storing a bioactive substance (30), and an aerosol generating device (16) connected to the reservoir (14) to receive the bioactive substance (30). The aerosol generating device (16) is configured for generating an aerosol comprising the bioactive substance (30) and for dispensing said aerosol into a respiration area in front of the mouth and nose of the user in order to provide a sterilising atmosphere therein. The invention further refers to a corresponding method of controlling a respiratory protection device.

### FIELD OF THE INVENTION

The present invention is in the field of medical devices. In particular, the invention refers to a head-mounted respiratory protection device configured for protecting a wearing user from inhaling and/or exhaling infectious agents by dispensing an aerosol. The invention further refers to a method of controlling a corresponding respiratory protection device.

### BACKGROUND OF THE INVENTION

Respiratory protection devices are worldwide used for preventing users from being exposed to haphazardous substances such as toxic gases via inhalation (environmental protection) and/or from exposing others to own-produced haphazardous substances such as infectious fluids via exhalation or ejection of respiratory droplets (subjective protection).

Millions of workers are for example required to wear respirators in industries having close contact to airborne contaminants, such as mining, metallurgy and chemical production. Such respirators are typically devices configured for filtering the surrounding air before it is inhaled by the user and/or to provide an independent non-contaminated oxygen supply. These devices have a complex structure, notable weight and volume and high production costs.

Lighter versions of respiratory protection devices are protection filtering masks such as FFP-masks and chirurgical masks used by medicine professionals. Such light devices still have the disadvantage of covering the face of the wearer, thereby impairing interpersonal communication, for example by screening the voice of the user and by preventing non-verbal communication.

The advent of new large-scale infectious diseases with rapid worldwide propagation, such as the SARS outbreak initiated in 2002, the MERS outbreak initiated in 2012 and -most notable—the Covid-19 pandemic started in 2020, have raised awareness towards the importance of having respiratory protection devices that are easy to produce and use and effective for avoiding the propagation of infectious diseases. In view of the aforementioned disadvantages of the prior art, there is however room for technical improvement in the field of respiratory protection devices.

### SUMMARY OF THE INVENTION

The present invention aims at solving the problem of providing a novel type of respiratory protection device overcoming the aforementioned disadvantages of the prior art. The invention refers in particular to a respiratory protection device according to claim 1, to a method of controlling a respiratory protection device according to claim 26, and to a computer-readable storage medium according to claim 31. Preferred embodiments of the invention are defined in the appended dependent claims.

The respiratory protection device according to the invention comprises a body-wearable elongated mount configured for attaching the respiratory protection device to the head and/or neck of a user. The respiratory protection device may hence in particular be a head-mounted device or neck-mounted device or both a head and neck-mounted device.

The elongated mount has a fixation end and a dosing end. The elongated mount may have an elongated body extending between the fixation end and the dosing end, wherein the elongated body may in some embodiments extend beyond the fixation end and/or beyond the dosing end.

The fixation end is attachable to the head and/or neck of the user. For example, the fixation end may have a curved shape, for example a hook-like shape, configured for being attached around an ear of the user. Thus, the respiratory protection device may be configured as an ear-mounted device.

However, the fixation end may have other configurations for attaching the respiratory protection device to the head and/or neck of the user. For example, the fixation end may comprise an elastic element, such as an elastic band, configured for being fitted around the head and/or neck of the user and providing supporting tension for holding the respiratory protection device. Additionally or alternatively, the fixation end may comprise an attaching mechanism, such as a clipping mechanism, a snapping mechanism or the like, configured for attaching the respiratory protection device to other head-held items such as eyewear, for instance protection glasses or conventional glasses, to a protection mask, a headset or a headband, allowing attaching the respiratory protection device to other head-held items worn by the user.

The elongated mount is shaped such that, when the respiratory protection device is attached to the head and/or neck of the user at the fixation end, the dosing end is arranged in a proximity of a respiration area located at least in part in front of the mouth and nose of the user. The respiration area may refer herein to a volume around the mouth and nose of the user from which air can be directly inhaled by the user and/or to which air can be exhaled by the user, in particular when breathing normally or when coughing. The exact location and size of the respiration area may however depend on the morphology and breathing capabilities of the user. In any case, the respiration area comprises at least an area surrounding the mouth and nose of the user and a close proximity thereof, for example an area comprising points in space within a distance of up to 30 cm, preferably up to 15 cm, more preferably up to 10 cm from at least the mouth or the nose of the user.

The mount may be shaped such that the dosing end of the elongated mount is arranged next to the respiration area or is pointing towards the respiration area when the respiratory protection device is attached to the head and/or neck of the user by the fixation end. The mount, in particular the dosing end thereof may or may not protrude from the surface of the users face. In some embodiments, the elongated mount may for example be configured for extending on the skin of the user, for example along a cheek of the user, and the dosing end may also be arranged on the cheek of the user and may protrude from the surface of the users face being arranged next to the mouth or the nose of the user pointing towards the respiration area and separated from the mouth and nose of the user by an air gap of up to 30 cm, preferably up to 15 cm, more preferably up to 10 cm.

In some embodiments, the mount may be of a bendable material, preferably of a shape-retaining bendable material. The bendable material may allow mechanically modifying the shape of the elongated mount such that the mount defines and preferably retains a new shape, thereby allowing adjusting a position and orientation of the dosing end of the mount, in particular with respect to the respiration area. This may further allow adapting the form of the mount to the face morphology of the user, for example by improving a fit of the mount to the head and/or neck of the user. The bendable material may comprise any suitable known skin-tolerant material used in head-sets, head-phones, hearing-aid etc., such as one or more of a metal or metal alloy such as nitinol, and/or a plastic material, such as thermoplastic material and any combination thereof. For example, a thermoplastic with supporting metal or metal alloy structures embedded.

The respiratory protection device further comprises a reservoir configured for storing a bioactive substance and an aerosol generating device connected to the reservoir to receive a bioactive substance stored in the reservoir. For example, the bioactive substance may be a fluid, such as a liquid solution, in which case the aerosol generating device may be fluidly connected to the reservoir, for instance by means of one or more connection hoses configured for transmitting the bioactive substance from the reservoir to the aerosol generating device. In another example, the bioactive substance may be a powder, which may be conveyed through the fluid connection, for instance through the connection hoses in a similar way as the liquid solution.

The aerosol generating device is arranged at the dosing end of the elongated mount and is configured for generating an aerosol comprising the bioactive substance and for dispensing said aerosol into the respiration area. Notably, the respiration area may have an effective size that effectively varies depending on the velocity at which the aerosol is dispensed (e.g. ejected) from the aerosol generating device.

When the respiratory protection device is attached to the head and/or neck of the user, the fixation end may be positioned above the dosing end and/or above the aerosol generating device. If the reservoir is arranged at the fixation end above the dosing end and/or above the aerosol generating device, the force of gravity may be used for conveying the bioactive substance from the reservoir to the aerosol generating device.

However, the respiratory protection device may alternatively or additionally comprise a pumping device configured for pumping the bioactive substance from the reservoir to the aerosol generating device. The pumping device includes means for conveying powder, such as pneumatic powder conveyors or mechanic powder conveyors.

The invention hence provides a respiratory protection device having a compact and simple design based on the localised administration of a bioactive substance into the respiration area around the mouth and nose of the user by means of an aerosol. The action of the bioactive substance, which may in particular comprise a bioactive agent such as an antibacterial and/or antiviral agent, may have several health-preserving effects: the bioactive substance comprised in the aerosol may allow providing a localised disinfecting effect in the respiration area, i.e. in a close proximity of the external access to the airways of the user, i.e. to the mouth and the nose of the user. When the aerosol generating device dispenses the aerosol into the respiration area, the aerosol forms a localised protective atmosphere around the mouth and nose of the user. This protective atmosphere can prevent the user from being infected with airborne bacteria, pathogens and/or viruses during inhalation.

Further, during inhalation, the protective atmosphere created by the aerosol in the respiration area can be partly inhaled by the user. Thereby, the bioactive substance can access the airways of the user through their mouth and/or nose and deposit therein, in the mouth, nose and/or upper airways of the user, forming a protective layer of bioactive substance which may prevent infection with inhaled infectious agents. For example, the aerosol may be smaller than 5 um, such that they reach lung tissue and pulmonary alveoli can act in anti-inflammatory and with healing effect.

In addition, the protective atmosphere created by the aerosol can also act as a purifying atmosphere through which any fluids coming from within the airways, mouth and/or nose of the patient flow during exhalation must cross, thereby undergoing disinfection.

Contrary to traditional respiratory protection devices, the respiratory protection device of the invention needs not completely cover the mouth and nose of the user and is hence more user-friendly, eliminating the asphyxia sensation characteristic of respiratory protection devices that do cover the mouth and nose of the user and not interfering in verbal and non-verbal communication, such as speaking or smiling.

According to some embodiments, the respiratory protection device may further comprise a control unit for controlling the aerosol generating device, such that the aerosol generating device dispenses the aerosol comprising the bioactive substance into the respiration area when activated by the control unit. The control unit thereby allows operating the aerosol generating device in a controlled manner in order to optimise the disinfecting effects of the aerosol comprising the bioactive substance. The control unit may preferably be integrated within the mount.

In preferred embodiments of the invention, the respiratory protection device may further comprise a respiratory activity sensor configured for detecting respiratory activity, in particular exhalation activity and/or inhalation activity, in a surrounding environment of the respiratory protection device. The respiration area may be partly or entirely comprised in said surrounding environment of the respiratory protection device. The respiratory activity sensor may be configured for detecting respiratory activity within a detection range, for example respiratory activity of the user and/or respiratory activity of a person other than the user located within the detection range. This can be achieved with a pre-selected or adjustable sensitivity of the respiration activity sensor for the aerosol and/or a specific design of the inlet/outlet with specific aerodynamic properties. The detection range may be of up to 3 m, preferably up to 2 m, more preferably up to 1 m. The detection range may depend on presence of wind in the area of the user in a preferable way and may be even more than 3 m. For example, the respiratory activity sensor may have adjustable sensitivity through the control unit, such that the detection range can be coarsely adjusted by the user. Respiratory activity may refer herein to any action in the course of which fluids, in particular air and/or corporal fluids such as saliva and mucosa, may enter or exit the body of a person through the nose and/or the mouth. Respiratory activity may for example refer to any of exhalation activity, inhalation activity, speaking activity, breathing activity, sneezing activity, coughing activity, spitting activity or any combination thereof.

The respiratory activity sensor is operatively connected to the control unit and the control unit is further configured for activating the aerosol generating device when the respiratory activity sensor detects respiratory activity. Thus, the control unit is configured for activating the aerosol generating device in reaction to the respiratory activity sensor detecting respiratory activity in said surrounding environment of the respiratory protection device. Thereby, the control unit timely synchronises the activation of the aerosol generating device and hence the dispensing of the aerosol comprising the bioactive substance into the respiration area with the presence of respiratory activity detected by the respiratory activity sensor, such that the protective atmosphere is created by the bioactive substance when the aerosol is dispensed into the respiration area when it is most effective.

The respiratory activity sensor may be arranged at the dosing end of the elongated mount. The respiratory activity sensor may be removably or fixedly attached to the aerosol generating device.

In some embodiments, the control unit may further be configured for controlling one or more aerosol parameters of the dispensing of the aerosol comprising the bioactive substance by the aerosol generating device. The one or more aerosol parameters may comprise a quantity of dispensed bioactive substance, a dispensing duration time, a dispensing intensity and/or a dispensing directionality. By controlling the quantity of dispensed bioactive substance, the control unit may determine how much bioactive substance is comprised in the aerosol dispensed into the respiration area and/or how much aerosol is dispensed into the respiration area. The control unit can be configured to operate the aerosol generating device for dispensing a predetermined quantity of bioactive substance and/or of aerosol every time the aerosol generating device is activated by the control unit, for example a quantity mass determined as fraction from a predetermined total mass of bioactive substance of 5 to 250 mmol, preferably 10 to 100 mmol and the estimated number of activations within a period of 2 to 6 hours. By controlling the dispensing duration time, the control unit may determine for how long the aerosol is dispensed by the aerosol generating device upon each activation. For example, the control unit can be configured to operate the aerosol generating device for dispensing the aerosol for a predetermined duration time of 0.1 s to 5 s, preferably from 0.5 s to 2 s every time the aerosol generating device is activated by the control unit. By controlling the dispensing intensity, the control unit may determine a velocity at which the aerosol is dispensed (ejected) into the respiration area, thereby indirectly determining the reach of the dispensed aerosol (the effective size of the respiration area).

For example, the control unit can be configured to operate the aerosol generating device for dispensing the aerosol with a velocity from 0.5 m/s to 5 m/s, preferably from 1.5 m/s to 4.5 m/s. By controlling the dispensing directionality the control unit may determine in which direction the aerosol is dispensed. For example, the control unit may be configured to operate the aerosol generating device for dispensing the aerosol towards the mouth and/or nose of the user and/or in a direction away from the mouth and/or nose of the user.

According to some embodiments, the control unit may further comprise or be connectable to a processing unit configured for analysing and/or classifying the detected respiratory activity. The processing unit may be configured for detecting one or more of exhalation activity, inhalation activity, speaking activity, breathing activity, sneezing activity, coughing activity and spitting activity. The processing unit may alternatively or in addition be configured for detecting and classifying artefacts, e.g. caused through wind-noise turbulent airflows of non-human nature. The respiratory activity sensor may generate a detection signal when respiratory activity is detected and the processing unit may be configured whether the detected respiratory activity corresponds to one or more of the aforesaid types of respiratory activity based on the detection signal, for example by comparing the detection signal to pre-stored table values or by means of a machine learning algorithm previously trained on a large number of detection signals for identifying respiratory activity as one or more of the aforesaid types of respiratory activity. The detection signal may comprise information about an audio recording of the respiratory activity and/or about one or more air flow parameters associated to the respiratory activity, such as air flow velocity, air flow humidity, air flow directionality and/or airflow quantity.

The control unit may further be configured for controlling the one or more aerosol parameters according to an analysis result and/or a classification result obtained by the processing unit. The settings of the aerosol parameters controlled by the controlled unit for dispensing the aerosol may hence vary depending on the results of the analysis and/or classification by the processing unit. For example, the processing unit may be configured for classifying a detected respiratory activity as breathing inhalatory activity if an air flow is detected flowing in a direction towards the mouth and/or nose of the user and having an air humidity below 90% and the control unit may be configured for setting the quantity of dispensed bioactive substance to 50 mmol, the dispensing duration time to 1 s and the dispensing intensity to 1.6 m/s if the processing unit detects breathing inhalatory activity. Following with this example, the processing unit may be configured for classifying a detected respiratory activity as coughing exhalatory activity if an air flow is detected flowing in a direction away from the mouth and/or nose of the user with an air velocity over 2 m/s and having an air humidity over 90% and the control unit may be configured for setting the quantity of dispensed bioactive substance to 300 mmol, the dispensing duration time to 3 s and the dispensing intensity to 4.5 m/s if the processing unit detects coughing exhalatory activity.

According to some embodiments, the aerosol generating device may further be configured for selectively dispensing the aerosol comprising the bioactive substance in a first direction and/or in a second direction different from the first direction, wherein the second direction might be opposite to the first direction. The first and second directions are thus different from each other and may in particular point away from each other. The aerosol generating device may for example comprise a first and a second set of nozzles for dispensing the aerosol in the first direction and in the second direction, respectively, wherein the first and second set of nozzles may be respectively oriented in the first and second directions. Throughout this description, a set of nozzles refers to one or more nozzles. The aerosol generating device may be positioned and oriented such that the first direction points towards the mouth and nose of the user and the second direction points away from the mouth and nose of the user when the respiratory protection device is attached to the head and/or neck of the user. The aerosol generating device may thus be configured for selectively dispensing the aerosol directed towards the mouth and nose of the user (in the first direction) and/or in a direction opposite to the mouth and nose of the user (in the second direction). Thus, the first and second set of nozzles may be configured such that, upon receiving a corresponding control instruction of the control unit, the aerosol generating device can selectively dispense the aerosol in the first direction towards the mouth and nose of the user through the first set of nozzles and/or away from the mouth and nose of the user in the second direction through the second set of nozzles. Additionally or alternatively, the aerosol generating device may comprise a selectively orientable dispensing head, for example a rotatable dispensing head, configured for selectively orienting according to a control instruction of the control unit.

The control unit may be adapted to selectively control, e.g. with or in part with user selectable parameters, the aforementioned dispensing directionality by controlling the activation of the first and second set of nozzles and/or the orientation of the orientable dispensing head.

In embodiments in which the mount is of a bendable material, the bendable properties of the mount may allow adjusting a position and/or an orientation of the dosing end of the mount in order to adjust a position and/or an orientation of the aerosol generating device, for example of the nozzles thereof through which the aerosol is ejected, such that the first direction is oriented towards the mouth and nose of the user and the second direction is oriented towards the respiration area away from the mouth and nose of the user.

In some embodiments, the control unit may be operatively connected to the reservoir and configured for monitoring one or more reservoir parameters. The one or more reservoir parameters may comprise a quantity of bioactive substance and/or a type of bioactive substance contained in the reservoir. The control unit may thereby detect what kind of bioactive substance is contained in the reservoir, for example what kind of bioactive agent the bioactive substance comprises, and/or how much of the bioactive substance is left in the reservoir. For this purpose, the reservoir may comprise or be connected to one or more reservoir sensors configured for measuring a filling stand of the reservoir and/or one or more parameters of the bioactive substance contained therein such as density, chemical composition and/or electrical conductivity and the control unit may be operatively connected to the one or more sensors.

According to some embodiments, the control unit may be connectable or connected to an external interface device, such as mobile phone, a smartwatch or a PC, and may be configured for transmitting to the external interface device the one or more aerosol parameters and/or the one or more reservoir parameters. The aforesaid processing unit may be comprised in the external interface device, for example in the form of a software tool. The control unit may thereby allow the external interface device to receive the one or more aerosol parameters and/or the one or more reservoir parameters for processing and/or displaying them. Additionally or alternatively, the control unit may be configured for receiving from the external interface device an input for adjusting the one or more aerosol parameters and/or an activation input triggering the activation of the aerosol generating device. The user may thereby be able to selectively adjust any of the aerosol parameters and/or to selectively trigger the activation of the aerosol generating device according to a control instruction received from the external interface device. For example, the user may manually trigger the activation of the aerosol generating device by pushing an activation button provided by a control software or app installed in the external interface device.

The respiratory activity sensor system may comprise one or more of an air pressure sensor, an air flow sensor, a temperature sensor, a proximity sensor and a humidity sensor. An air pressure sensor may be configured for detecting variations of air pressure in a surrounding environment of the sensor and may allow the respiratory activity sensor to detect the presence, intensity and/or directionality of detected respiratory activity. An air flow sensor may be configured for detecting a quantity and/or a velocity of air flowing through the air flow, in particular of air being inhaled or exhaled by the user, and may allow the respiratory activity sensor to detect a quantity and/or velocity of air of detected respiratory activity. A proximity sensor may be configured to detect the absence or presence of other persons within a range from the user. In addition, or alternatively, the proximity sensor may estimate the distance from the user to the other person closest to the user, through e.g. measurement of flight-time of an ultrasonic-pulse, infrared-pulse or other known measurement principles. A temperature sensor and a humidity sensor may be used by the respiratory activity sensor for detecting, respectively, a temperature and a humidity of the air flowing due to the respiratory activity. The information about the respiratory activity detected by the respiratory activity sensor can be used by the control unit for analysing and/or classifying the respiratory activity as previously explained. Further, the information about the respiratory activity detected by the respiratory activity sensor can be used by the control unit to recalibrate if necessary the one or more aerosol parameters and/or the settings of the aerosol generating device.

In preferred embodiments of the invention, the respiratory activity sensor may comprise a microphone. A microphone is a version of an air pressure sensor allowing the respiratory activity sensor to detect the presence, intensity and/or directionality of detected respiratory activity in the form of sound vibrations. The microphone may preferably be a piezo microphone, which allows the microphone to be particularly resistant against humidity, thereby improving a durability of the respiratory activity sensor. If the respiratory activity sensor comprises a microphone, respiratory activity may be detected in the form of respiratory sound.

When the respiratory activity sensor comprises a microphone, the respiratory protection device may preferably further comprise a sound processing device connected to the microphone and configured for generating a sound detection signal based on respiratory sound detected by the microphone and for transmitting the sound detection signal via control unit to an external device. The sound detection signal may be an electric signal, in particular an audio signal, in which information about the sound detected by the microphone is coded and/or compressed. The sound processing device may in addition or alternatively be configured for voice recognition from the sound detection signal and generating from the recognized voice a sound command signal for transmission to the control unit. The control unit may, at least in part, control operation of the aerosol generating device based on the received sound command signal, e.g. stopping dispensing upon receiving a stop sound command signal or changing various dispensing parameters upon receipt of specific sound command signals, such as quantity of bioactive substance, dispensing velocity, dispensing direction and dispensing duration. It is understood, that a number of further voice-controlled commands useful for operating the respiratory protection device are imaginable and that the device is not limited to only the listed voice-commands above. Voice-control has the further advantage, that the respiratory protection device must not be touched with potentially infectious or otherwise contaminated hands of the user. The respiratory protection device may hence further operate as or be implemented in a conventional sound input device, for example a hands-free microphone and/or a headset for the external device. The external device may for example be a mobile phone or a PC, and may in particular correspond to the external interface device referred to above, and/or a loudspeaker. The respiratory protection device may be connected to the external device by means of a wired connection or a wireless connection such as Bluetooth, or WiFi. The respiratory protection device of the invention may hence be implemented in a conventional audio input device such as a headset or a hands-free microphone. Existing audio input devices can be adapted to implement a respiratory protection device according to the invention.

Different configurations of the reservoir are foreseen:

According to some embodiments, the reservoir may be at least partially integrated within the mount, preferably at the fixation end. This provides a particularly compact design.

The reservoir may comprise a valve for filling bioactive substance into the reservoir and/or for extracting bioactive substance from the reservoir in a controlled manner. The reservoir may for example be a cavity formed within the fixation end of the mount configured for receiving and storing the bioactive substance therein.

In some embodiments, the reservoir may be removably or fixedly attached to the mount, preferably at the fixation end. The For example, the reservoir may comprise a capsule, a bottle or a tank configured for being removably or fixedly attached to the mount. The mount may comprise a valve mechanism for providing a connection between the reservoir and the aerosol generating device. If the reservoir is removably attached to the mount, the reservoir may preferably be a replaceable reservoir.

In some embodiments, the reservoir may be at least partially integrated within the aerosol generating device and/or removably or fixedly attached to the aerosol generating device. This way, a distance between the reservoir and the aerosol generating device can be reduced, such that the bioactive substance may be more easily conveyed from the reservoir to the aerosol generating device. This can be particularly advantageous for high-density fluid bioactive substances and for powder-based bioactive substances. The reservoir may for example be an exchangeable capsule removably attached to the aerosol generating device, allowing to replace an empty capsule by a full capsule and/or exchanging the type of bioactive substance if necessary.

In some embodiments, the aerosol generating device may be removably attached to the mount, preferably at the dosing end, and the reservoir may be integrated within the aerosol generating device, such that the aerosol generating device can be configured as a one-use device that can be easily removed together with the reservoir for disposal after use. Preferably, the respiratory activity sensor may then be attached to the mount, such that the respiratory activity sensor needs not be replaced when the aerosol generating device is replaced. However, in other embodiments, the aerosol generating device may be fixedly attached to the mount.

In some embodiments, the reservoir may be an independent reservoir connected to the aerosol generating device by means of one or more connection hoses for transmitting the bioactive substance. The independent reservoir may be structurally independent from the rest of the respiratory protection device and needs not be configured for being attached to the head and/or neck of the user. Instead, the independent reservoir may be arranged elsewhere when the mount is attached to the head and/or neck of the user. For example, the independent reservoir may be a wearable independent reservoir configured for being attached to other parts of the body of the user such as the waist, the torso, the back or an extremity of the user or to other wearables or garments configured for being attached to the aforesaid parts of the body of the user. An independent reservoir may have a larger capacity and hence allow receiving and storing a larger amount of bioactive substance.

The reservoir may be an exchangeable reservoir. An exchangeable reservoir allows easily replacing an empty reservoir after its use by a new full reservoir and/or exchanging a reservoir containing one type of bioactive substance by a reservoir containing a different type of bioactive substance to adapt the respiratory protection device to new use requirements.

According to some embodiments, the reservoir may be a pressurisable reservoir. The bioactive substance may be a pressurised bioactive substance, for example a bioactive substance comprising a pressurising fluid acting as a propellant for generating the aerosol, and the reservoir may be configured for receiving and storing the pressurised bioactive substance. The pressurising fluid may include any suitable and known propellants and preferably comprise nitrogen-di-oxide N₂O (nitrous oxide) and/or oxygen O₂. The pressurising effect of the pressurising fluid or propellant can then be used to convey the bioactive substance from the reservoir to the aerosol generating device and/or to generate the flow of aerosol from the aerosol generating device into the respiration area.

According to some embodiments, the respiratory protection device may further comprise a bioactive substance contained within the reservoir. The bioactive substance may comprise a bioactive agent, preferably an antibacterial and/or antiviral agent, preferably n-chlorotaurine (C₂H₆ClNO₃S, PubChem CID: 108018). The bioactive substance may be a fluid substance, in particular a liquid substance, or a powder-based substance. In some embodiments, the bioactive substance may comprise one or more of an aromatising, a flavouring substance, a stabiliser substance and a preservative substance. The bioactive substance may be a fluid, preferably a liquid, or a powder.

In some embodiments, the mount may be a hollow mount defining a lumen therethrough, wherein one or more electrical connections, such as electrical wires, and/or fluid connections, such as connection hoses, between the aerosol generating device, the reservoir and/or the control unit may preferably be guided within the lumen.

In some embodiments, the lumen may be configured as a fluid connection between the aerosol generating device and reservoir, such that the inner walls of the mount defining the lumen limit a tubular channel through which the bioactive substance can flow between the reservoir and the aerosol generating device. Electrical wiring connecting the aerosol generating device with the reservoir and/or the control unit may be provided within the lumen or outside the lumen, in particular on an exterior surface of the mount.

The aerosol generating device may comprise or be a spraying device configured for spraying the aerosol comprising the bioactive substance, in particular when the bioactive substance is a liquid. The spraying device may comprise one or more spraying valves for controlling a fluid access of a pressurised bioactive substance from the reservoir to one or more spraying heads in which one or more spraying nozzles are formed. The one or more spraying valves may be configured as a one-way valve allowing the bioactive substance to flow only from the reservoir to the spraying head but not to flow back from a respective spraying head to the reservoir. The one or more spraying valves may be provided adjacent to the reservoir, for example at the fixation end of the mount, or adjacent to the spraying head, for example at a dosing end of the mount. An electrically or mechanically controlled actuator, preferably a piezo actuator, may be configured for controlling the one or more spraying valves, wherein the one or more spraying valve block an access of the bioactive substance to the respective spraying head in a closed state and allow the access of the bioactive substance to the respective spraying head in an open state. Each of the one or more spraying heads may comprise a set of nozzles configured for spraying the aerosol containing the bioactive substance. The nozzles may have a predetermined nozzle opening side determining a particle size or droplet size of the aerosol and/or a particle size distribution or droplet size distribution of the aerosol. The nozzles may be oriented in the spraying head such as to determine a directionality of the aerosol. For example, some of the nozzles may be oriented in a first direction, for example towards the mouth and nose of the user, and some of the nozzles may be oriented in a second direction, wherein the second direction is preferably opposed to the first direction, for example a second direction facing away from the mouth and nose of the user. In some embodiments, the nozzles may be formed using MEMS technology.

The aerosol generating device may comprise or be a nebuliser configured for nebulising the aerosol comprising the bioactive substance, in particular when the bioactive substance is a liquid. The nebuliser may comprise one or more nebulising chambers configured for receiving the bioactive substance from the reservoir and one or more vibrating elements arranged in the nebulising chamber and configured for vibrating to generate the aerosol comprising the bioactive substance, which is then ejected through a set of nozzles or openings formed in the nebuliser, in particular in a pinhole mask, due to the vibrating movement of the vibrating element. The vibrating element may be a piezoelectric vibrating element and the nebuliser may be configured as an ultrasonic nebuliser. Alternatively, the vibrating element may be a vibrating membrane element or a vibrating pinhole mask comprising the set of nozzles and the nebuliser may be configured as a vibrating mesh technology (VMT) nebuliser.

The aerosol generating device may comprise or be a powder aerosol generating device, preferably a dry powder aerosol generating device for dispensing the aerosol comprising the bioactive substance, in particular when the bioactive substance is a powder. The reservoir may be integrated within the powder aerosol generating device, for example in the form of a replaceable powder capsule or forming a cavity integrated within the powder aerosol generating device. The powder aerosol generating device may be configured for generating an air flow through the reservoir to generate the aerosol comprising the powder bioactive substance, for example by means of a ventilator or a vibrating actuator, in particular a vibrating piezo actuator. The powder aerosol generating device may comprise an air flow directing channel configured for directing an air flow of the generated air flow. The geometry and dimensions of the air flow directing channel may be adapted to the desired dynamics of the air flow for generating the aerosol. The air flow directing channel may have a tapered cross-section increasing from the interior of the aerosol generating device towards the exterior of the air flow directing channel. The powder aerosol generating device may comprise a permeable regulating membrane or pinhole mask configured for regulating a flow of aerosol from the aerosol generating device, for example a flow of aerosol out of the air flow directing channel. The permeable regulating membrane or pinhole mask may be configured for determining a particle size or droplet size of the aerosol and/or a particle size distribution or droplet size distribution of the aerosol.

According to some embodiments, the aerosol generating device may further comprise one or more flow directing structures for directing a flow of the bioactive substance dispensed by the aerosol generating device. The flow directing structures may preferably be configured for directing the flow of the aerosol in a first direction, for example towards the mouth and nose of the user, and/or in a second direction, preferably a second direction opposite to the first direction, for example away from the mouth and nose of the user. The flow directing structures may comprise fixed structures, such as one or more flow directing channels configured for directing the flow of the aerosol along a respective channel direction, or movable structures, such as orientable and/or rotatable wings, configured for directing the flow of the aerosol depending on a respective orientation of the movable structure. The aerosol generating device may for example comprise a first flow directing channel configured for directing the flow of aerosol in the first direction a second flow directing channel configured for directing the flow of aerosol in the second direction, towards a region located in front of the mouth and nose of the user. Additionally or alternatively, the aerosol generating device may comprise a plurality of orientable rotatable wings configured for directing the flow of aerosol towards the mouth and nose of the user in a first orientation and for directing the flow of aerosol away from the mouth and nose of the user in a second orientation, wherein the first and second orientations may respectively correspond to different rotation angles of the wings.

The present invention further refers to a method of controlling a respiratory protection device, in particular a respiratory protection device according to any of the previously described embodiments. The method may be a computer-implemented method implemented by the control unit and/or a processing unit of the respiratory protection device respectively corresponding to the previously described control unit and processing unit or by a processor in general. The controlled respiratory protection device may comprise an aerosol generating device for dispensing and aerosol comprising a bioactive substance into a respiration area located at least in part in front of the mouth and nose of a user and a respiratory activity sensor for detecting respiratory activity in a surrounding environment of the respiratory protection device.

The method comprises detecting respiratory activity in said surrounding environment of the respiratory protection device by means of the respiratory activity sensor and generating an activation signal for activating the aerosol generating device in response to the respiration activity sensor detecting respiratory activity. As a result, an activation signal is generated that can be used to activate the aerosol generating device upon detecting respiratory activity, such that the aerosol generating device dispenses the aerosol comprising the bioactive substance into the inhalation area when respiratory activity is detected, in a timely synchronised manner.

The method may further comprise analysing and/or classifying the respiratory activity detected and/or artefact detected by the respiratory activity sensor. The respiratory activity may be analysed and/or classified based on one or more air flow parameters associated to the respiratory activity, such as air flow velocity, air flow humidity, air flow directionality and/or airflow quantity. The analysing and/or classifying the respiratory activity may comprise detecting one or more of exhalation activity, inhalation activity, speaking activity, breathing activity, sneezing activity, coughing activity, spitting activity and any combination thereof. In order to analyse and/or classify detected respiratory activity as respiratory activity corresponding to any of the aforementioned types of respiratory activity, the detected respiratory activity may be compared to table values comprised in a pre-stored table storing measured or simulated parameters corresponding to different types of respiratory activity. Additionally or alternatively, the detected respiratory activity may be analysed and/or classified by a machine learning algorithm trained on measured or simulated respiratory activity for identifying the detected respiratory activity as respiratory activity corresponding to any of the aforementioned types of respiratory activity.

Detecting the respiratory activity may comprise detecting an air flow corresponding to the respiratory activity. The detected air flow may be analysed by determining the air flow velocity, the air flow humidity, the air flow directionality and the air flow quantity of the air flow and comparing it to pre-stored table values to determine which combination of pre-stored values best resembles the detected combination of air flow parameters. Additionally or alternatively, the detected respiratory activity may be analysed by analysing a detected sound recording of the respiratory activity. By means of any of the aforementioned analysis and/or classification techniques, it may be for example be determined that the detected air flow corresponds to sneezing activity of the user or to coughing activity of a person other than the user.

The method may further comprise generating or modulating the activation signal for controlling one or more aerosol parameters of the activation signal for controlling the dispensing of the aerosol by the aerosol generating device according to an analysis result and/or a classification result. The one or more aerosol parameters may comprise a quantity of dispensed bioactive substance, a duration time of the dispensing, a dispensing intensity and a dispensing directionality or any combination thereof. For example, the method may comprise modulating the activation signal for controlling a quantity of dispensed bioactive substance and whether the bioactive substance is dispensed towards the mouth and nose of the user or away from it depending on the result of the analysis. For instance, if the analysis determines that a detected air flow corresponds to sneezing activity of the user, the method may comprise generating an activation signal containing control instructions for the aerosol generating device that cause the aerosol generating device to adjust the one or more aerosol parameters to correspond to a first combination of aerosol parameters associated to sneezing activity of the user and, if the analysis determines that a detected airflow corresponds to coughing activity of a person other than a user, the method may comprise generating an activation signal containing control instructions for the aerosol generating device that cause the aerosol generating device to adjust the one or more aerosol parameters to correspond to a second combination of aerosol parameters associated to coughing activity of a person other than the user, wherein said second combination may be different from the first combination. For example, according to the first combination of aerosol parameters, the aerosol may be dispensed in a direction away from the mouth and nose of the user with a highest possible dispensing intensity (in order to maximise the reach of the aerosol), and according to the second combination of aerosol parameters, the aerosol may be dispensed both in a direction away from the mouth and nose of the user and towards the mouth and nose of the user with a dispensing intensity lower than the highest possible dispensing intensity.

Analysing and/or classifying the respiratory activity detected by the respiratory activity sensor may comprise detecting whether the respiratory activity originates from a user of the respiratory protection device, wherein the respiratory protection device is attached to the head and/or neck of the user, or from a person other than the user situated in an environment of the user.

In some embodiments, the aerosol generating device may further be configured for selectively dispensing the aerosol directed in a first direction, towards the mouth and nose of a user wearing the respiratory protection device, wherein the respiratory protection device is attached to the head and/or neck of the user, and/or in a second direction opposite to the mouth and nose of the user. Detecting the respiratory activity may then comprise detecting whether the detected respiratory activity corresponds to exhalation activity or inhalation activity and detecting whether the respiratory activity originates from the user or from a person other than the user situated in an environment of the user. Detecting the respiratory activity may further comprise detecting whether the detected respiratory activity corresponds to any of speaking activity, breathing activity, sneezing activity, coughing activity, spitting activity and any combination thereof. The activation signal may be generated for selectively controlling the aerosol generating device to dispense the aerosol according to one or more different schemes, in particular according to any combination of the first to fourth schemes described below, depending on whether the detected respiratory activity corresponds to exhalation or inhalation activity and on whether the detected respiratory activity corresponds to respiratory activity of the user or of a person other than the user:

A first scheme may comprise selectively controlling the aerosol generating device to dispense the aerosol directed towards the mouth and nose of the user when exhalation activity of a person other than the user is detected by the respiratory activity sensor, in particular when said exhalation activity corresponds to coughing, spitting or sneezing activity. The first scheme may hence be configured for protecting the user wearing the respiratory protection device from being infected by bacterial and/or viral agents expelled by said person other than the user in case of inhaling said agents by dispensing the aerosol comprising the bioactive substance towards the mouth and nose of the user, thereby providing a disinfecting effect in the mouth and nose of the user and/or in a near environment thereof.

A second scheme may comprise selectively controlling the aerosol generating device to dispense the aerosol in said direction opposite to the mouth and nose of the user when exhalation activity of the user of the respiratory protection device is detected by the respiratory activity sensor, in particular when said exhalation activity corresponds to coughing, spitting or sneezing activity. The second scheme may hence be configured for preventing infectious bacterial and/or viral agents produced by the user from dispersing into an environment of the user by dispensing the aerosol comprising the bioactive substance into the respiration area in a timely synchronised manner with exhalation activity of the user, thereby providing a disinfecting effect in front of the mouth and nose of the user.

A third scheme may comprise selectively controlling the aerosol generating device to dispense the aerosol directed towards the mouth and nose of the user when inhalation activity of the user of the respiratory protection device is detected by the respiratory activity sensor, in particular when said inhalation activity corresponds to breathing. The third scheme may hence be configured for timely synchronising inhalation activity of the user with dispensing the aerosol towards the mouth and nose of the user so as to provide the disinfecting effect of the bioactive substance therein, thereby protecting the user from being infected during inhalation, for example in densely-crowded areas.

A fourth scheme may comprise selectively controlling the aerosol generating device to dispense the aerosol in said direction opposite to the mouth and nose of the user when inhalation activity of a person other than the user is detected by the respiratory activity sensor, in particular when said inhalation activity corresponds to breathing activity. The fourth scheme may thereby be configured for timely synchronising inhalation activity of said person other than the user with dispensing the aerosol away from the mouth and nose of the user, i.e. towards said person, in particular when said person is close to the user, so as to provide the disinfecting effect of the bioactive substance therein, thereby protecting said person from being infected during inhalation.

The present invention further refers to a computer-readable storage medium comprising instructions which, when executed by a processor, cause the processor to carry out the method of controlling a respiratory protection device according to any of the previous described embodiments.

### BRIEF DESCRIPTION OF THE FIGURES

FIG. 1 shows a schematic illustration of a respiratory protection device according to an embodiment of the invention.

FIG. 2 shows a schematic illustration of a respiratory protection device according to another embodiment of the invention.

FIG. 3 shows a schematic illustration of a respiratory protection device according to another embodiment of the invention.

FIG. 4 schematic illustrates the working principle of a respiratory protection device according to the invention.

FIG. 5 is a flow diagram schematically illustrating a method of controlling a respiratory protection device according to embodiments of the invention.

### DETAILED DESCRIPTION OF PREFERRED EMBODIMENTS OF THE INVENTION

For the purposes of promoting an understanding of the principles of the invention, reference will now be made to a preferred embodiment illustrated in the drawings, and specific language will be used to describe the same. It will nevertheless be understood that no limitation of the scope of the invention is thereby intended, such alterations and further modifications in the illustrated apparatus and such further applications of the principles of the invention as illustrated therein being contemplated as would normally occur now or in the future to one skilled in the art to which the invention relates.

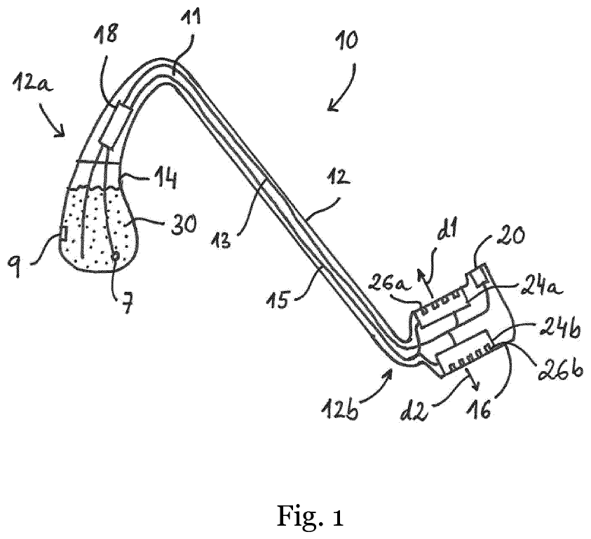

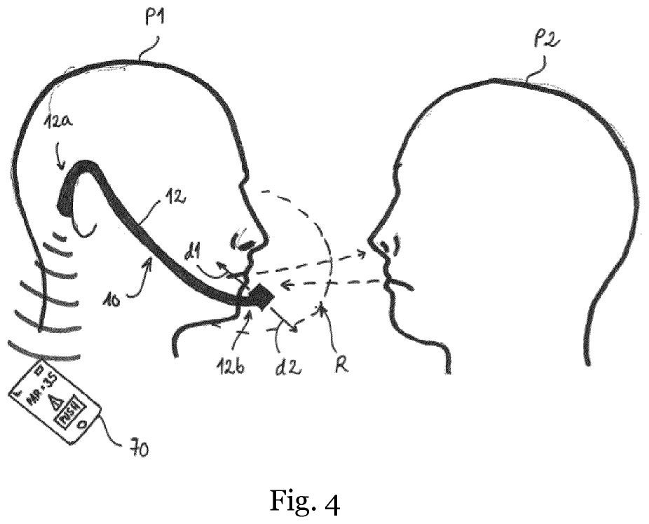

FIGS. 1 to 3 show schematic illustrations of exemplary embodiments of the respiratory protection device 10. These figures are best understood in combination with FIG. 4, showing a schematic illustration of the working principle of a respiratory protection device 10 worn by a user P1. Throughout the figures, the same reference numerals are used for identifying the same elements.

FIG. 1 shows a schematic illustration of a respiratory protection device 10 according to embodiments of the present invention. The respiratory protection device 10 comprises an elongated mount 12 that extends from a fixation and 12a to a dosing end 12b. The fixation end 12a of the elongated mount 12 is configured for being attached to the head and/or neck of a user of the respiratory protection device 10. In the embodiment shown, the fixation end 12a has a hook-like shape adapted for being attached to the ear of the user by lying on and around the upper part of the ear flap of the patient (see also FIG. 4). The straight section of the elongated mount 12 has a length of about 15 cm.

As shown in FIG. 4, the mount 12 is shaped such that, when the respiratory protection device 10 is attached to the head and/or neck of the user P1 by the fixation end 12a, the mount 12 extends along the face of the patient and the dosing end 12b is arranged below the fixation end (considered in the vertical direction) and in a proximity of a respiration area R located in front of the mouth and nose of the user P1. When the user P1 inhales, air from the respiration area R may enter through the mouth and nose of the user P1. When the user P1 exhales, air may be expelled from the mouth and nose of the user P1 into the respiration area R.

As shown in FIG. 1, the respiratory protection device 10 further comprises a reservoir 14 configured for storing a bioactive substance 30. In the embodiment shown in FIG. 1, the bioactive substance is a liquid substance comprising a liquid antiviral bioactive agent pressurised with O₂. The reservoir 14 is integrated within the mount 12, formed as a cavity at the fixation end 12a of the mount 12 and configured for receiving the bioactive substance 30. The reservoir 14 comprises a valve 9 for refilling the reservoir 14 with bioactive substance 30 and/or for extracting bioactive substance from the reservoir 14 when necessary.

The respiratory protection device 10 further comprises an aerosol generating device 16 arranged at the dosing end 12b and fixedly attached thereto. The aerosol generating device 16 is connected to the reservoir 14 by a connection hose 15 that establishes a fluid connection between the reservoir 14 and the aerosol generating device 16. The connection hose 15 is guided through a lumen 11 formed within the elongated mount 12.

The aerosol generating device 16 can receive the bioactive substance 30 stored in the reservoir through the connection hose 15 and is configured for generating an aerosol comprising the bioactive substance 30 and for dispensing the aerosol into the respiration area R shown in FIG. 4.

The respiratory protection device 10 further comprises a control unit 18 that is integrated within the mount 12 and is configured for controlling the aerosol generating device 16. The aerosol generating device 16 dispenses the aerosol into the respiration area R when activated by the control unit 18 by means of a corresponding control instruction transmitted through an electric wire 13 by means of which the control unit 18 is connected to the aerosol generating device 16. Like the connection hose 15, the electric wire 13 is guided through the lumen 11 formed in the interior of the elongated mount 12.

The control unit 18 is further connected to a reservoir sensor 7 arranged in the reservoir 14 and configured for detecting a filling level of the reservoir 14. When the reservoir 14 is empty or the biological substance 30 is below a predefined filling level, the control unit 18 can produce a corresponding warning signal for letting the user know that the reservoir 14 has to be refilled with biological substance through the valve 9.

The respiratory protection device 10 further comprises a respiratory activity sensor 20 arranged at the dosing end 12b of the mount 12 and fixedly attached to the aerosol generating device 16. The respiratory activity sensor 20 is configured for detecting respiratory activity in a surrounding environment of the respiratory protection device 10, for example respiratory activity in the respiration area R illustrated in FIG. 4. The respiratory activity sensor 20 is operatively connected to the control unit 18 by means of the electric wire 13. When the respiratory activity sensor 20 detects respiratory activity, a detection signal is sent to the control unit 18 transmitting information about the detected respiratory activity. The control unit 18 is configured for activating the aerosol generating device 16 in response to said detection signal sent by the respiratory activity sensor 20, i.e. when the respiratory activity sensor 20 detects respiratory activity.

The aerosol generating device 16 of the embodiment shown in FIG. 1 is configured as a spraying device configured for generating an aerosol comprising the pressurised liquid bioactive substance 30 contained in the reservoir 14 and for spraying the aerosol into the respiration area R. The spraying device 16 comprises a first spraying head 24a comprising a first set of nozzles 26a oriented in a first direction di and a second spraying head 24b comprising a second set of nozzles 26b oriented in a second direction d2. The first direction d2 and the second direction d2 are opposite to each other.

The elongated mount 12 is made of a bendable shape-retaining material that allows adjusting a position and orientation of the aerosol generating device 16 such that the first direction d1 points towards the mouth and nose of the user P1 and the second direction d2 points away from the mouth and nose of the user P2 as shown in FIG. 4.

The spraying device 16 of FIG. 1 comprises a first one-way spraying valve (not shown in FIG. 1) and a second one-way spraying valve (not shown in FIG. 1) for controlling the access of the biological substance 30 into the spraying heads 24a and 24d of the spraying device 16, respectively, through the connecting hose 15. Each spraying valve is opened and closed by a piezo actuator that is electrically controlled by the control unit 18. When the control unit 18 sends an activation signal to the spraying device 16 through the electrical wire 13, the piezo actuator opens the respective spraying valve and thereby causes the pressurised bioactive substance 30 to be sprayed into the respiration area R in the form of an aerosol.

The control unit 18 can selectively activate the spraying device 16 for spraying the aerosol in the first direction d1 through the first set of nozzles 26a of the first spraying head 24a and/or in the second direction d2 through the second set of nozzles 26b of the second spraying head 24b. Thereby, the control unit 18 can selectively control whether the aerosol is dispensed in the first direction d1 towards the mouth and nose of the user P1 and/or in the second direction t2 away from the mouth and nose of the user P1 (see FIG. 4).

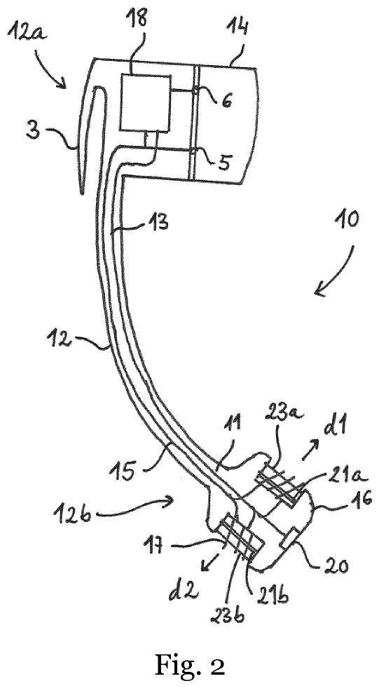

FIG. 2 shows a further embodiment of a respiratory protection device 10 according to the invention having basically the same structure as the embodiment shown in FIG. 1. However, in the embodiment shown in FIG. 2, the fixation end 12a, instead of having a hook-like shape allowing the respiratory protection device 10 to be worn by a user as an ear-held device, comprises a clipping structure 3 configured for attaching the fixation end 12a to a band that the user may wear around their head, for example a headband or an elastic band of a protective eyewear item, or to the temple of an eyewear item worn by the user.

A further difference of the embodiment represented in FIG. 2 with respect to the embodiment represented in FIG. 1 is that the reservoir 14 of the embodiment shown in FIG. 2 is provided as a replaceable capsule configured for being removably attached to the mount 12 at the fixation end 12a. A valve mechanism 5 is provided at an interface between the mount 12 and the reservoir 14 through which the bioactive substance 30 can flow from the reservoir to the aerosol generating device 16 over the connection hoe 15 in a controlled manner. The valve mechanism 5 is controlled by the control unit 18. Further, a reservoir sensor 6 is provided at said interface between the mount 12 and the reservoir 14 and allows the control unit 18 to monitor a quantity of bioactive substance 30 and a type of bioactive substance contained in the reservoir 14. The bioactive substance 30 is, in the embodiment shown in FIG. 2, a liquid substance containing an antiviral and antibacterial agent pressurised with NO.

In the embodiment shown in FIG. 2, the aerosol generating device 16 is a VMT nebuliser comprising a nebulising chamber formed in the interior of the nebuliser 16 (not shown in FIG. 2). When the bioactive substance 30 flows from the reservoir 14 to the nebuliser 16, it is received in the nebulising chamber. The nebuliser 16 further comprises first and second vibrating membranes 21a and 21b configured for vibrating when activated by the control unit 18 to generate the aerosol, which is then nebulised into the respiration area. When the first vibrating membrane 21a is activated, the aerosol is nebulised in the first direction d1 through openings formed in a first pinhole mask 23a. When the second vibrating membrane 21b is activated, the aerosol is nebulised in the second direction d2 through openings formed in a second pinhole mask 23b. The first and second pinhole masks 23a, 23b determine, through the size and number of their openings, a droplet size and size distribution of the aerosol. The nebuliser further comprises orientable wings 17 that are orientable with different angles to direct the flow of the aerosol.

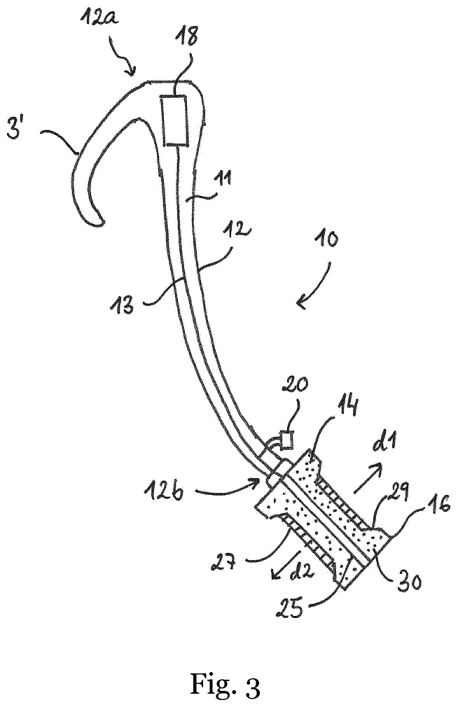

FIG. 3 shows a further embodiment of a respiratory protection device 10 according to the invention having basically the same structure as the embodiment shown in FIG. 1, wherein the fixation end 12a also has a hook-like shape allowing the respiratory protection device 10 to be worn by a user as an ear-held device.

However, in the embodiment shown in FIG. 3, the aerosol generating device 16 is configured as a replaceable one-use device that is removably attached to the dosing and 12b of the mount 12. The reservoir 14 is formed by an interior cavity of the aerosol generating device 16 and contains the bioactive substance 30, which in this embodiment is a powder-based substance, although in other embodiments it can be a liquid substance. Other than in the embodiment shown in FIGS. 1 and 2, the respiratory activity sensor 20 is directly attached to the dosing end 12b of the mount 12 and not to the aerosol generating device 16.

In the embodiment shown in FIG. 3, the aerosol generating device 16 is a powder aerosol generating device that is configured for generating the aerosol by creating an air flow through the reservoir 14 by means of a babe vibrating rating movement of a piezoelectric vibrating actuator 25 that is driven by the control unit 18. When the control unit 18 sends an activation signal to the aerosol generating device 16 through the electrical wire 13, the piezoelectric vibrating actuator 25 vibrates and generates an air flow that carries away part of the powder contained in the reservoir 14 within the aerosol generating device 16, thereby generating the aerosol. The aerosol is dispensed in the first and second directions d1, d2 through openings formed in respective permeable regulating membranes 27. The permeable regulating membranes 27 determine, through the size and number of their openings, a droplet size and size distribution of the aerosol. The flow of the aerosol is directed in the first and second directions d1, d2 by respective tapered channels 29, which have a cross-section that increase from an interior to an exterior of the aerosol generating device 16.

As illustrated in FIG. 4, the control unit 18 of the respiratory protection device 10 is connectable, by means of a wireless connection, such as Bluetooth or Wi-Fi, to an external interface device 70, in this case a smart phone. A control app installed in the external interface device 70 allows the user P1 wearing the respiratory protection device 10 to use the external interface device 70 as an input device for manually setting one or more aerosol parameters such as dispensing intensity or dispensing time duration, for reading a quantity of bioactive substance 30 left in the reservoir 14 as detected by the control unit 18, and for manually triggering the activation of the aerosol generating device 16. Further, the respiratory activity sensor 20 of any of the embodiment shown in FIGS. 1 to 3 may be or comprise a microphone, and the respiratory protection device 10 can operate as an audio input device for the external interface 70, for example as a conventional hands-free microphone for having a telephone conversation using the smart phone 70.

As shown in FIG. 4, in a proximity of the user P1, there is another person P2 other than the user.

In any of the embodiments shown in FIGS. 1 to 3, the respiratory activity sensor 20 can comprise an air pressure sensor, an air flow sensor and a humidity sensor for detecting air flows associated to respiratory activity and for determining a velocity, flow quantity and a humidity of the corresponding air flow.

Based on the velocity, flow quantity and a humidity detected by the respiratory activity sensor 20, a processing unit, which can be a processing unit integrated in the control unit 18 or in the external interface device 70, analyses the detected respiratory activity and allows the control unit 18 to generate an activation signal for activating the aerosol generating device 16 in a manner adapted to the detected respiratory activity.

The processing unit is configured for determining whether the detected respiratory activity corresponds to respiratory activity of the user P1 or of the other person P2 (cf. FIG. 4) and whether it corresponds to exhalation activity, inhalation activity, speaking activity, breathing activity, sneezing activity, coughing activity, spitting activity and any combination thereof.

Based on the type of respiratory activity determined by the processing unit, the control unit 18 of the respiratory protection device 10 generates an activation signal according to a corresponding signal generation scheme for activating the aerosol generating device, respectively, according to a different aerosol generation scheme. For each signal generation scheme, the activation signal may be configured for causing the aerosol generating device to dispense the aerosol according to a different combination of: quantity of dispensed bioactive substance, duration time of the dispensing, dispensing intensity and dispensing directionality.

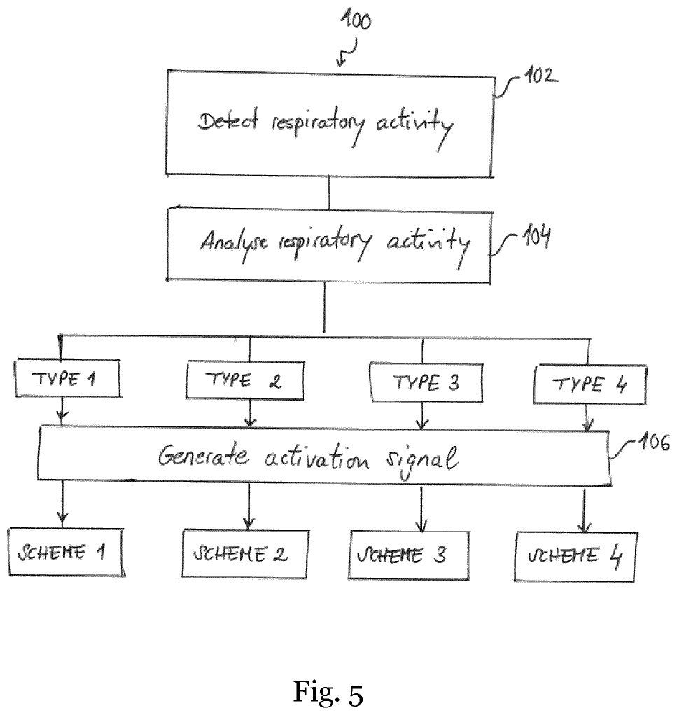

FIG. 5 shows a schematic flow diagram illustrating a corresponding method 100 of controlling the respiratory protection device 10 in the situation represented in FIG. 4.

If the user P1 or the other person P2 produces an air flow corresponding to respiratory activity, this is detected at 102 by the respiratory activity sensor 20, which determines the velocity, flow quantity and humidity of the corresponding air flow.

The processing unit analyses, at 104, the velocity, flow quantity and humidity of the air flow detected by the respiratory activity sensor 20, and based thereon, determines whether the detected respiratory activity corresponds to respiratory activity of one of different types of respiratory activity TYPE 1, TYPE 2 TYPE 3 or TYPE 4. TYPE 1 corresponds to exhalation coughing activity of the person P2, TYPE 2 corresponds to exhalation sneezing activity of the user P1, TYPE 3 corresponds to inhalation breathing activity of the user P1 and TYPE 4 corresponds to inhalation breathing activity of the person P2.

The activation signal is generated at 106 by the control unit 18 according to a particular signal generation scheme depending on the type of respiratory activity to which the detected respiratory activity is associated. For respiratory activity associated to TYPE 1, TYPE 2 TYPE 3 or TYPE 4, the activation signal is respectively generated according to a corresponding signal generation scheme SCHEME 1, SCHEME 2, SCHEME 3 or SCHEME 4.

An activation signal according to SCHEME 1 is configured for causing the aerosol generating device 16 to dispense a quantity Q1 of bioactive substance, during a dispensing time T1, with a dispensing intensity V1 and in the first direction d1.

An activation signal according to SCHEME 2 is configured for causing the aerosol generating device 16 to dispense a quantity Q2>Q1 of bioactive substance, during a dispensing time T2>T1, with a dispensing intensity V2>V1 and in the second direction d2.

An activation signal according to SCHEME 3 is configured for causing the aerosol generating device 16 to dispense a quantity Q1 of bioactive substance, during a dispensing time T3>T1, with a dispensing intensity V3<V1 and in the first and second directions d1 and d2.

An activation signal according to SCHEME 4 is configured for causing the aerosol generating device 16 to dispense a quantity Q2 of bioactive substance, during a dispensing time T2, with a dispensing intensity V1 and in the second direction d2.

The aforementioned types of respiratory activity and signal generation schemes are exemplary. Other combinations are possible.

Although preferred exemplary embodiments are shown and specified in detail in the drawings and the preceding specification, these should be viewed as purely exemplary and not as limiting the invention. It is noted in this regard that only the preferred exemplary embodiments are shown and specified, and all variations and modifications should be protected that presently or in the future lie within the scope of protection of the invention as defined in the claims.

1. A respiratory protection device comprising:
a body-wearable elongated mount for attaching the respiratory protection device to the head and/or neck of a user, wherein the mount has a fixation end and a dosing end, wherein the fixation end is attachable to the head and/or neck of the user, and wherein the elongated mount is shaped such that, when the respiratory protection device is attached to the head and/or neck of the user at the fixation end, the dosing end is arranged in a proximity of a respiration area located, at least in part, in front of a mouth and nose of the user;
a reservoir for storing a bioactive substance;
an aerosol generating device connected to the reservoir to receive the bioactive substance stored in the reservoir, wherein the aerosol generating device is arranged at the dosing end of the elongated mount, wherein the aerosol generating device is configured for generating an aerosol comprising the bioactive substance and for dispensing the aerosol into the respiration area;
a control unit for controlling the aerosol generating device, such that the aerosol generating device dispenses the aerosol comprising the bioactive substance into the respiration area when activated by the control unit; and
a respiratory activity sensor configured for detecting respirator activity in a surrounding environment of the respiratory protection device, wherein the respiratory activity sensor is operatively connected to the control unit,
wherein the control unit is further configured for activating the aerosol generating device when the respiratory activity sensor detects the respiratory activity and configured for controlling one or more aerosol parameters for the aerosol generating device that dispenses the aerosol comprising the bioactive substance, wherein the one or more aerosol parameters include a quantity of dispensed bioactive substance, a duration time of dispensing the aerosol, a dispensing intensity, a dispensing directionality, or combinations thereof.

2-4. (canceled)

5. The respiratory protection device of claim 1, wherein the control unit further comprises or is connectable to a processing unit configured for analyzing and/or classifying the detected respiratory activity, and wherein the control unit is further configured for controlling the one or more aerosol parameters according to an analysis result and/or a classification result obtained by the processing unit.

6. The respiratory protection device of claim 1, wherein the aerosol generating device is further configured for selectively dispensing the aerosol comprising the bioactive substance in a first direction and/or in a second direction opposite to the first direction.

7. The respiratory protection device of claim 1, wherein the control unit is further operatively connected to the reservoir and configured for monitoring one or more reservoir parameters, wherein the one or more reservoir parameters comprise a quantity of bioactive substance and/or a type of bioactive substance contained in the reservoir.

8. The respiratory protection device of claim 1, wherein the control unit is connectable or connected to an external interface device and is configured for transmitting to the external interface device the one or more aerosol parameters and/or the one or more reservoir parameters, and/or is configured for receiving from the external interface device an input for adjusting the one or more aerosol parameters and/or an activation input triggering the activation of the aerosol generating device.

9. The respiratory protection device of claim 1, wherein the respiratory activity sensor comprises an air pressure sensor, an air flow sensor, a temperature sensor, a proximity sensor, a humidity sensor or combinations thereof.

10. The respiratory protection device of claim 1, wherein the respiratory activity sensor comprises a microphone.

11. The respiratory protection device of claim 10, further comprising a sound processing device connected to the microphone, wherein the sound processing device is configured for generating a sound detection signal based on sound detected by the microphone and for transmitting the sound detection signal to an external device.

12. (canceled)

13. The respiratory protection device of claim 1, wherein the reservoir is:
at least partially integrated within the mount at the fixation end;
removably or fixedly attached to the mount at the fixation end;
at least partially integrated within the aerosol generating device;
removably or fixedly attached to the aerosol generating device at the fixation end; and
an independent reservoir connected to the aerosol generating device by means of one or more connection hoses for transmitting the bioactive substance.

14. The respiratory protection device of claim 1, wherein the reservoir is an exchangeable reservoir.

15. The respiratory protection device of claim 1, wherein the reservoir is a pressurizable reservoir.

16. (canceled)

25. The respiratory protection device of claim 1, wherein the aerosol generating device comprises one or more flow directing structures for directing a flow of the aerosol dispensed by the aerosol generating device, wherein the flow directing structures are configured for directing the flow of the aerosol in a first direction and/or in a second direction opposite to the first direction.

26. A method of controlling a respiratory protection device, the respiratory protection device comprising:
an aerosol generating device for dispensing an aerosol comprising a bioactive substance into a respiration area located, at least in part, in front of the mouth and nose of a user; and
a respiratory activity sensor for detecting respiratory activity in a surrounding environment of the respiratory protection device,
wherein the method comprises:
detecting respiratory activity in said surrounding environment of the respiratory protection device by means of the respiratory activity sensor, and
generating an activation signal for activating the aerosol generating device in response to the respiratory activity sensor detecting respiratory activity, and
analyzing and/or classifying the respiratory activity detected by the respiratory activity sensor,
wherein the activation signal is generated for controlling one or more aerosol parameters for the aerosol generating device that dispenses the aerosol according to an analysis result and/or a classification result, wherein the one or more aerosol parameters include a quantity of dispensed bioactive substance, a duration time of dispensing the aerosol, a dispensing intensity, a dispensing directionality, or combinations thereof.

27-28. (canceled)

29. The method of claim 26, wherein the analyzing and/or classifying the respiratory activity detected by the respiratory activity sensor comprises detecting one or more of exhalation activity, inhalation activity, speaking activity, breathing activity, sneezing activity, coughing activity, spitting activity or any combination thereof.

30. The method of claim 26, wherein the analyzing and/or classifying the respiratory activity detected by the respiratory activity sensor comprises detecting whether the respiratory activity originates from a user of the respiratory protection device, wherein the respiratory protection device is attached to the head and/or neck of the user, or from a person other than the user situated in an environment of the user.

31-32. (canceled)

33. The respiratory protection device of claim 1, wherein the reservoir is at least partially integrated within the mount at the fixation end.

34. The respiratory protection device of claim 1, wherein the reservoir is at least removably or fixedly attached to the mount at the fixation end.

35. The respiratory protection device of claim 1, wherein the reservoir is at least partially integrated within the aerosol generating device.

36. The respiratory protection device of claim 1, wherein the reservoir is at least removably or fixedly attached to the aerosol generating device at the fixation end.

37. The respiratory protection device of claim 1, wherein the reservoir is at least an independent reservoir connected to the aerosol generating device by means of one or more connection hoses for transmitting the bioactive substance.
